(19) United States
(12) Patent Application Publication
(10) Pub. No.: US 2008/0090900 A1
(43) Pub. Date: Apr. 17, 2008

(54) ANTIBIOTIC BUSHRIN

(75) Inventors: Nuzhat Ahmed, Karachi (PK); Bushra Uzair, Karachi (PK); Viqar Uddin Ahmad, Karachi (PK); Farzana Kousar, Karachi (PK)

Correspondence Address: SARFARAZ K. NIAZI, 20 RIVERSIDE DRIVE, DEERFIELD, IL, 60015, US

(73) Assignee: University of Karachi, Karachi (PK)

(21) Appl. No.: 11/548,715

(22) Filed: Oct. 12, 2006

(51) Int. Cl. A61K 31/341; A61P 31/04; C07D 307/02; C12P 21/04

(52) U.S. Cl. 514/461; 435/71.3; 549/498

(57) ABSTRACT

This invention describes a new antibiotic, hereinafter designated as bushrin, having the formula C₁₆H₁₆O₂, which is 7-(3-furyl)-3,7-dimethyl-7,8-dihydro-1-naphthalenol, its production method by fermentation, and the methods related to its recovery, concentration and purification from the crude solutions. The invention includes within its scope the agent in dilute form, as a crude concentrate, and in pure form. The invention also relates to the use of the compound according to the invention in antimicrobial compositions and as antiseptics or disinfectants.

This invention discloses isolation, identification and demonstration of lytic antibiotic activity of a novel antibiotic against Gram-positive and Gram-negative bacteria, and which is stable at temperature ranging from 30 to 100° C. and within the pH range from 2 to 7. The novel antibiotic designated as bushrin (Compound 1), was obtained from the marine bacterium, Pseudomonas stutzeri, isolated from the Ribbonfish of the Baluchistan coast of Pakistan. The antibiotic was present in the ethyl acetate extract of the growth media and had a chemical structure of 7-(3-furyl)-3,7-dimethyl-7,8-dihydro-1-naphthalenol (Compound 1), which was highly effective against Salmonella typhi, Vibrio cholera, Proteus mirabilis, Hafnia alvei, Serratia marcescens, Bacillus subtilis, Staphylococcus aureus, Staphylococcus epidermidis, Enterobacter faecalsi: the activity being higher than generally associated with antibiotics vancomycin, tetracycline, ampicillin and nisin

### BACKGROUND

Antibiotics are widely found in nature including the marine environment, which encompasses almost three quarters of the surface of the planet and it is estimated that more than 80% of marine life forms remain unexplored. Although the chemical compounds derived from marine microorganism sources are less well known than those derived from the terrestrial sources are, several bioactive compounds have been reported during the last decade making the marine environment a fertile source of new drugs. (Kasanah, N, Hamann, M T 2004. Development of antibiotics and the future of marine microoganisms to stem the tide of antibiotic resistance. Curr Opin Investig Drugs.5 (8): 827-37).

Marine microorganisms have physiological properties distinct from those of terrestrial microorganisms and are thus these are regarded as potential new source of biologically active substances (Okami, Y. Marine microorganisms as a source of bioactive substance. 1993. P. 651-655. In M. J. Klung (Ed), Current perspective in microbial ecology. American Society for Microbiology, Wash., D.C.). Competition for limiting natural resources within a microbial community is thought to be an important selective force that promotes synthesis of antimicrobial compounds (Fenton, A. M., P. M. Stephens, J, Crowley, M. O'Callaghan and F O'Gara (1992) Exploitation of gene(s) involved in 2,4-diacetylphloroglucinol biosynthesis to confer a new biocontrol capability to Pseudomonas strain 58:3873-3878.) The production of antimicrobial metabolites is a complicated process involving various factors e.g., substrate availability and the physiological state of organism. Many strains, which do not produce antibiotics in pure culture, can be induced to do so by exposing them to living cells. Induction of antibiotic production has also been shown for endosymbiotic bacteria and even for human pathogens such as Bacillus subtilis, Escherichia coli and Pseudomonas aeroginosa. Screening of marine epibiotic bacteria isolated from surface of marine algae and invertebrates has shown that a high percentage of them produce antimicrobial metabolites (Mearns-Spragg, A., Bregu, M., Boyd, K. G & Burgess, J. G. 1988. Cross-species induction and enhancement of antimicrobial activity produced by epibiotic bacteria from marine algae and invertebrates, after exposure to terrestrial bacteria. Letters in Applied Microbiology 27, 142-146; Burgess, J. G, Mearns-Spragg, A., Boyd, K. G, & Bregu, M. 1999 Microbial antagonism: A new avenue in natural products research. Journal of Biotechnology 70, 27-32). A large number of surface-associated marine bacteria have also been found to produce antibiotics. Trischman et al., (Trischman, J. A, Tapiolas, D. M, Jensen, P. R., D. Wigght, R. Fenical, W., Mckee T. C., Ireland, C. M., Stout, T. Jclardy 1994. Salinamides-A and Salinamidew B-infamatory dipeptides from a marine Streptomyces. J. AM. Chem. Soc 116,757-758,) isolated a species of Streptomyces from surface of a Jellyfish, which produced two bicyclic peptides (salinamides A and B). These compounds have novel backbones and exhibit activity against a wide array of Gram-positive bacteria. Antibiotics from free-living marine microorganisms have also been reported, including loloatins from Bacillus, Agrochelin and sesbanimides from Agrobacterium (Acebal, C., L. M. Cañedo, J. L. F. Puentes, J. P. Baz, F. Romero, F. De La Calle, M. D. G. Grávalos, and P. Rodrigues.1999. Agrochelin, a new cytotoxic antibiotic from a marine Agrobacterium. Taxonomy, fermentation, isolation, physicochemical properties and biological activity. J. Antibiot. 52:983-987), pelagiomicins from Pelagiobacter variabilis (Imamura, N., M. Nishijima. T. Takadera, K. Adachi, M. Sakai, and H. Sano.1997. New anticancer antibiotics, pelagiomicins produced by a new marine bacterium Pelagiobacter variabilis. J. Antibiot. 50:8-12), indomycinone from a Streptomyces species (Biabani, M. A. F., H. Laatsch, E. Helmke, and H. Weyland. 1997. Indomycinone: a new member of pluramycin class of antibiotics isolated from marine Streptomyces sp. J. Antibiot. 50:874-877) and dihydrophenocomycin methyl ester from Streptomyces (Pusecker, K., H. Laatsch, E. Helmke, and H. Weyland. 1997. Dihydrophencomycin methyl ester, a new phenazine derivative from a marine Streptomycete. J. Antibiot, 50:479-483).

Thus, marine bacteria can provide novel antimicrobial compounds. However, in order to find more novel structures new ways of screening for those compounds must be applied. In this invention, we have isolated a marine bacterium, producing antibacterial compound. Phenotypic characterization and API 20NE (Biomerieux automated 24-48 hour identification of gram-negative non-Enterobacteriaceae) analysis suggests that strain CMG 1030 is Pseudomonas stutzeri and we have purified the antibacterial substance and determined its chemical structure and evaluated its antibacterial and bactericidal activities against Gram-positive and Gram-negative bacteria.

Materials and Methods

Clinical isolates were collected from various medical centers of Pakistan and United States and quality control strains were obtained from the American type culture (ATCC). Identification of each bacterial culture was done by conventional methods: Gram-negative rods were identified with API20E and 20NE system and Staphylococci with Staph Trac system (BioMerieux). All bacterial strains were stocked in trypticase soy broth (TSB medium) anaerobic strains were stocked in brain heart infusion broth containing 20% glycerol. The bacteria were slanted on the respective media before being used in the experiments. Vancomycin, ampicillin, nisin, tetracycline were purchased from Sigma Chemical Company (St. Louis, Mo.). Column chromatography (CC) was performed on silica gel (E. Merck, 70-230mesh) TLC: pre-coated silica gel G-25-UV 254; detection at 254 nm. Optical rotation was recorded on Jasco-Dip-360 digital polarimeter. UV and IR spectra were recorded on Hitichi-UV-3200 and Jasco-320-A spectrophotometers respectively; UV in nm (log ε) and cm⁻¹, resp. ¹H-NMR and ¹³C-NMR Spectra were recorded on Bruker AM-400 and AMX-500(for 2D) spectrophotometers; SiMe₁ was used as an external standard; δ in ppm, J in Hz EI- and C1-MS; JMS-HX-110 with a data system.

Fermentation, Isolation and Purification of Antibacterial Compound

A seed culture was prepared and inoculated onto King B agar medium plates and were incubated at 30° C. for 5 days. The culture was first extracted with 80% acetone arid then with ethyl acetate from the agar surface at room temperature. The combined ethyl acetate extract was evaporated under vacuum to yield the crude ethyl acetate extract (5 g). The crude ethyl acetate extract was again extracted with hexane, chloroform, ethyl acetate respectively. The ethyl acetate extract (1.95 g) was subjected to CC over silica gel column using hexane with gradient of ethyl acetate up to 100%. Pseudomonas stutzeri grew well when it was cultured to produce Compound 1 on King B medium and was confluent on day 5. The antibacterial activity was detected in ethyl acetate fraction of crude extract of Pseudomonas stutzeri grown in agar plates indicating that the antibacterial substance(s) might not be bound on the cell surface; further isolation was performed by partition of ethyl acetate extract with chloroform. Methanol and water. A total of 5 g crude extract was isolated from 5 Lt culture. An antibacterial substance Compound 1 (4 mg) was purified by silica gel column chromatography.

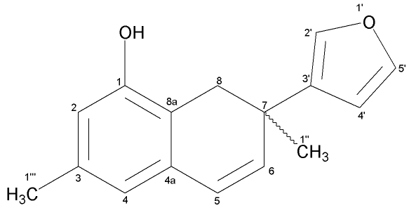

Insert FIG. 1

Description of FIG. 1: Chemical Structure of Antibiotic Bushrin

Compound 1

Physical Properties

Compound 1 was yellow powder with molecular formula of C₁₆H₁₆O₂, EI-MS (m/z)=208,161.9,136.9, 109; UV λₘₐₓin CHCl₃(nm)=248,193,363; IR vₘₐₓ(cm⁻¹)=13241,2922,1619.2; soluble in methanol, chloroform and ethyl acetate: insoluble in water and butanol.

Antibacterial Activity

The disc diffusion technique (Bauer A. W, Kirby W, M. M, Sherrisc J C, Turck, M Antibiotic susceptibility testing by a standardized single disc method. Am J Clin Path 1966; 45:493-6) was used to determine the antibacterial activity of Compound 1 against clinical and environmental isolates. Sterile discs containing 150 μg of compound were prepared from a 10 mg/mL stock solution of compound in dimethyl sulfoxide. The Mueller Hinton (MH Oxoid) agar plates were seeded with test organism the prepared discs were placed on to the centre of plates and incubated at 37° C. for 24 hours. Compound 1 was active against Salmonella typhi. Vibrio cholera, Proteus mirabilis, Hafnia alvei, Serratia marcesens, Bacillus subtilis, Staphylococcus aureus (MRSA &MSSA), Staphylococcus epidermids, Enterobacter faecalis but less active against anaerobic bacterium Clostridium sporogenes and Clostridium perfringens. It is not active against Candida albicans.

Minimal Inhibitory Concentration

The minimum inhibitory concentration (MIC) of Compound 1 was determined by the standard microdilution method described in the national committee for clinical laboratory standards (National Committee for Clinical Laboratory Standards, 1997. Method for dilution antimicrobial susceptibility test for bacteria, that grow aerobically, 4th ed., p, 1-29. National Committee for Clinical Laboratory Standards) using Muller Hinton broth medium (Oxoid) incubated at 37° C. for 24 h. The MIC of Compound 1 for Staphylococcus aureus, Staphylococcus epidermidis were 50-100 μg/ml for Enterobacter faecalis 100 μg/ml and for Bacillus subtilis, Bacillus steriothermophilus was 50-60 μg/ml, for Salmonella typhi was 75 μg/ml, for Vibrio cholera, 60 μg/ml, for Proteus mirabilis 75 μg/ml, for Hafnia alvei 100 μg/ml for Serratia marcescens 60 μg/ml. However Compound 1 was less active against anaerobic bacteria Clostridium sporogenes and Clostridium perfringenes and the MIC was 150-200 μg/ml respectively. This substance was slightly active against Streptococcus sp for which the MIC was 200 μg/ml. This substance was not found active against Candida albicans. Table 1 shows the in vitro activity of Compound 1 against environmental and clinical isolates.

TABLE 1
In vitro activities of Compound 1 against
environmental and clinical isolates

| | | | MIC |
|---|---|---|---|
| Organism | Test Strains | Sensitivity | (μg/ml |
| Gram positive bacteria | MSSA methicillin sensitive | ++++ | 60 |
| | staphylococcus aureus | | |
| | MRSA methicillin resistant | ++++ | 60 |
| | staphylococcus aureus | | |
| | Staphylococcus epidermidis | ++++ | 50 |
| | Enterococcus faecalis | ++++ | 50 |
| | Enterococcus faecium | ++++ | 50 |
| | Bacillus subtilis | ++++ | 50 |
| | Bacillus steriothermophilus | ++++ | 50 |
| | Bacillus cereus | ++++ | 60 |
| | Streptococcus group G | − | — |
| | Clostridium perfringens | ++ | 100 |
| | Clostridium sporogenes | ++ | 125 |
| Gram negative bacteria | Escherichia coli | ++ | 75 |
| | Vibrio harveyi | ++++ | 75 |
| | Salmonella typhi | +++ | 75 |
| | Proteus morgani | ++++ | 75 |
| | Proteus mirabilis | ++++ | 75 |
| | Serratia marcescens | +++ | 60 |
| | Shewanella putrifacians | ++ | 75 |
| | Hafnia alvei | +++ | 100 |
| | Klebsiella aerogenes | − | — |
| Yeast | Candida albicans | − | — |

^{a}sensitivity test by disc diffusion technique
^{b}Width of inhibition zone: ++++ 18 to 30 mm; +++ 15 to 20; ++ 10 to 15; − no inhibition
^{c}MIC minimal inhibitory concentration.

Heat Stability Test

Solution stock of Compound 1 in methanol was diluted with water, and then heated at 30, 40, 50,60,70,80and 90° C. using a thermostat and at 100° C. by boiling for 10 mm. After the treatment, the liquid in each tube was evaporated completely and Compound 1 was re-dissolved in methanol to give an appropriate concentration. Then antibacterial activity was evaluated in triplicate by the disk diffusion method. The antibacterial activity of Compound 1 was stable between 30 and 100° C. but its antibacterial activity significantly reduced after treatment at 120° C. Activity was completely lost at 200° C.,

Bacteriolytic Assay

The seed culture of Staphylococcus aureus in Luria broth (LB) were washed twice with sterile distilled water containing 0.9% sodium chloride and the absorbance was adjusted to 0.05 at 600 nm. The bacterial cell suspension was divided into aliquots of 5 ml each placed in sterile test tube and exposed to Compound 1 at various concentration, untreated bacterial suspension were used as the negative control. These test tubes were incubated at 30° C. by shaking at 120 rpm the absorbance was measured 0, 60, 120, 180, 240, 300, 360 min. Reduction of the absorbance of the Staphylococcus aureus cell suspension was observed in the presence of Compound 1, in contrast the absorbance was reduced early in the incubation period. These results indicated that Compound 1 lyse Staphylococcus aureus.

Time Kill Experiment

The time kill experiment was conducted by the method described by Aeschlimann and Rybak (Aeschlimann, J. R., and M. J. Rybak. 1998. Pharmacodynamic analysis of the activity of quinopristin-dalfopristin against vancomycin-resistant Enterococcus faecium with differing MBCs via time-kill-curve and post antibiotic effect methods. Antimicrob. Agents Chemother. 42:2188-2192). The experiment was conducted in 50 ml Erlenmeyer flasks containing 25 ml of fresh nutrient broth (Oxoid) inoculated with overnight culture of Staphylococcus aureus to give an initial density of 106 cells/ml. The inoculation was carried out immediately after addition of antibacterial compound or test antibiotic at final concentrations that consisted of the MIC, two and four times the MIC. The flasks were further incubated at 30° C. with stirring with a magnetic stirrer at 200 rpm. The viable cell count of Staphylococcus aureus was estimated at various incubation times by the plating method. To minimize the effect of antibiotic carryover the samples were centrifuged at 1600×g for 5 min then the antibiotic medium was replaced with fresh nutrient broth. The cells were re-suspended and plated on to nutrient agar plates, the plates were incubated at 37° C. for 24 h, and colonies were counted. The time kill experiment showed that Compound 1 was able to decrease the counts of Staphylococcus aureus (ATCC33591) at MIC 50 μg/ml. This substance decreased the number of viable bacterial cells alter 8 hr of exposure. A decrease in bacterial cell counts was more readily found when the strains were exposed to Compound 1 at higher concentration. At two times the MIC and four times the MIC the viable count decreased drastically after 2 hand 1 h where as at two and for times of MIC of vancomycin, ampicillin, tetracycline the bacterial cell count decreased gradually as compare to Compound 1 however Compound 1 decreased the bacterial viable counts more significantly than vancomycin, nisin, tetracycline and ampicillin at the same concentration and after the same length of exposure, indicating that the killing rate of Compound 1 for laboratory strain of Staphylococcus aureus (ATCC33591) was much higher then those of test antibiotics.

Spheroplasting Activities

The ability of Compound 1 and known cell wall active antibiotics to induce the formation of osmotically fragile spheroplast was tested in a growing culture of Staphylococcus aureus. Aliquots (100 μl) of an exponential phase culture(A_{600,}0.05) grown in Luria broth supplemented with 0.3M concentration of sucrose were dispensed into test tubes containing different concentration of Compound 1 and 1 μl/ml ampicillin as positive control. The tubes were then incubated at 30° C. with shaking at 140 rpm. After 90 minutes, the cells were examined by phase contrast microscope for any morphological changes or for the presence of spheroplast. To confirm the osmotic fragility of the spheroplast the lytic effect of 2-3 μl of water added under the cover slip was tested. A log phase culture of Staphylococcus aureus growing in osmotically protected medium supplemented with 0.3M sucrose was treated with various concentration of Compound 1. Compound 1 did not induce formation of osmotically fragile spheroplasts similar to those observed with ampicillin. This observation suggests that the bacterial cell wall might not be the site of action of Compound 1.

Phase contrast Microscopy

An exponential phase culture (A₆₀₀, 0.05) of Bacillus subtilis and Staphylococcus aureus grown in Luria broth was treated with 50 μl/ml of Compound 1 for Bacillus subtilis and 60 μl/ml for Staphylococcus aureus and slides were immediately made in 1% agarose using 4 μl of Compound 1 treated cultures, time lapse images were obtained under phase contrast microscope, particular care was taken to minimize the sample exposure to UV light, image grabbing were performed essentially as described by Edwards and Errrington. (Edwards, D. H., and Errington, J.(1997). The Bacillus subtilis DIvIVA protein targets to the division septum and control the site specificity of cell division. Mol Microbiol 24:905-915). Antibiotic treated cells of Staphylococcus aureus and Bacillus subtilis were observed under phase contrast microscope and time lapse images were captured where a uniform and consistent decrease in size of cells of Bacillus subtilis were observed immediately after treatment with Compound 1. When cells of Staphylococcus aureus were treated with this compound 1 complete lyses was observed immediately alter treatment.

Chemical Structure of Compound 1

The compound 1 trivially named as 7-(3-furyl)-3,7-dimethyl-7,8-dihydro-1-naphthalenol was isolated from ethyl acetate soluble fraction of Pseudomonas stutzrei. The molecular formula C₁₆H₁₆O₂ of Compound 1 was deduced from HR-EIMS at m/z=240.2712. This compound has one aromatic, one cyclohexene and one furane ring. The ¹H-NMR spectrum displayed four aromatic protons. The doublet at δ 8.27(d, J=1.2) was meta coupled to the proton at δ 8.34 (dd, J=1.2, 1.4) in ¹H NMR spectrum and belongs to the protons (H-2) and (H-4) respectively. The cyclohexene ring showed two vicinal protons in ¹H-NMR spectrum at δ 8.44(d, J=8.8, H-5) and at δ 8.42 (d, J=8.8, H-6) respectively. The furan moiety was confirmed by its downfield vicinal proton appearing in ¹H-NMR spectra at δ 7.97(dd, J=1.2, 5.9, H-4′) and δ 8.84 (d, J=6.6, H-5′) respectively, while the methylene proton of cyclohexene ring appeared at δ 1.2 s each of these proton showed their singlets in ¹H-NMR spectrum. The singlet for the protons of two methyls (C-1″) and (C-1′″) appeared in ¹H-NMR spectra at δ 1.37and δ 2.26 respectively. The DEPT experiment displayed sixteen carbon signals out of which seven are methines, two methyles, and one methylene and six quaternary carbons, while the HMBC, HMQC and COSY data confirmed that the structure of Compound 1 is 7-(3-furyl)-3,7-dimethyl-7,8-dihydro-1-naphthalenol. Table 2 lists the NMR spectroscopic parameters of Compound 1.

TABLE 2
NMR data of compound 1(75MHz for ¹³C, 400MHz for
¹H-NMR in CDCl₃)

| POSITION | ¹H NMR | ¹³CNMR |
|---|---|---|
| 1 | — | 144.5 |
| 1′ | — | — |
| 1″ | 1.37 s | 14.3 |
| 1′″ | 2.26 s | 23.6 |
| 2 | 8.27(d, J=1.2) | 128.1 |
| 2′ | 8.02(d, J=1.2) | 132.1 |
| 3 | — | 142.09 |
| 3′ | — | 142.1 |
| 4 | 8.34(dd, J=1.2, 1.4) | 130.2 |
| 4′ | 7.97(d, J=1.2, 5.9) | 133.3 |
| 4 a | — | 143.2 |
| 5 | 8.44(d, J=8.8) | 135.1 |
| 5′ | 8.84(d, J=6.6) | 136.2 |
| 6 | 8.42(d, J =8.81) | 136.1 |
| 7 | — | 38.01 |
| 8 | 1.2 s | 30.7 |
| 8 a | | 144.3 |

a Coupling constants in Hertz are given in parenthesis

### DETAILED DESCRIPTION

During the lass 10 years, marine microorganisms have provided a large number of new natural products which have been derived from microorganisms. Since Burkholder (Burkholder. P. R P, Fisher, and F. Leitz. 1966. Production of pyrrole antibiotic by a marine bacterium. Appl. Microbiol. 14: 649-653) determined the first structure of a novel antibiotic produced by marine bacteria, there have been growing interest in marine bacteria as a potential source of natural products of pharmaceutical importance. This paper describes the fermentation, purification and elucidation of the chemical structure of antibacterial compound. Compound 1 produced by a marine bacterium active against clinical and environmental isolates. This bacterium was isolated from the gut of ribbonfish caught from Baluchistan coast of Pakistan. Pseudomonas stutzeri is a gram negative pigmented bacterium that produces antibacterial compound. The main product of this strain is an antibacterial compound designated as Compound 1, derived from ethyl acetate extract of the bacterial cells. In this study Compound 1 was determined to be 7-(3-furyl)-3,7-dimethyl-7,8-dihydro-1-naphthalenol and this substance is a new naturally occurring substance. This is also the first report of a marine Pseudomonas that produces this antibacterial compound. Antibacterial aid antifungal compounds by fluorescent Pseudomonas strains have been reported (Fenton, A. M, P. M. Stephens, J. Crowley, M. O'Callaghan arid F O'Gara (1992) Exploitation of gene(s) involved, in 2,4-diacetylphloroglucinol biosynthesis to confer a new biocontrol capability to Pseudomonas strain 58:3873-3878) such as 2,4-Diacetylphloroglucinol(DAPG) produced by Pseudomonas has drawn attention in the medical area because of bacteriolytic activity of DAPG against multi drug resistant Staphylococcus aureus (Isnansetyo, A., and Y. Kamei. 2003. A bactericidal antibiotic produced by a new marine bacterium, Pseudomonas phenolica sp.nov.O-BC30 T, against methicillin-resistant Staphylococcus aureus. Antimicrob. Agents Chemother.47:480-488).

This compound 1 gave excellent activity against Gram-positive bacteria then Gram-negative bacteria and produced very large zones of inhibition and minimal inhibitory concentration (MIC) for Gram-positive bacteria was between 50-75 μg/ml whereas for Gram negative bacteria it was between 75-125 μg/ml. It has been reported that DAPG (2, 4 diacetyl phloroglucinol) produced by Pseudomonas sp AMSN has high in vitro anti-MRSA activity comparable with vancomycin (Isnansetyo, A., M, Horikwa and Y, Kamei. (2001) In vitro antimethicillin resistant Staphylococcus aureus activity of 2, 4, diacetylphloroglucinol produced by Pseudomonas sp. AMSN Isolated from a marine alga. Journal of Antimicrobial Chemotherapy 47:719-730).

Compound 1 demonstrates activity against Salmonella typhi, Vibrio cholera, Proteus mirabilis, Hafnia alvei, Serratia marcescens, Bacillus subtilis, Staphylococcus aureus (MRSA &MSSA), Staphylococcus epidermidis, Enterobacter faecalis but less active against anaerobic bacterium Clostridium sporogenes and Clostridium perfringens. It is not active against Candida albicans.

The time kill experiment indicated that Compound 1 is rapidly bactericidal against Staphylococcus aureus. The killing rate of Compound 1 exhibited concentration dependent bactericidal activity and its killing rate was faster with an increase in the concentration and slower rate was observed at lower concentration. Compound. 1 at 125 μg/ml was able to kill Staphylococcus aureus completely and at 100 μg/ml it completely killed Bacillus subtilis. Compound 1 also killed cells of Staphylococcus aureus in osmotically protected medium which suggest that cell wall might not be the site of action.

What is claimed is:

1. The compound which has the structure represented by the formula C₆H₁₆O_{2,} which is 7-(3-furyl)-3,7-dimethyl-7,8-dihydro-1-naphthalenol [Compound 1];
[Insert FIG. 1)
Compound 1

2. A method of treating a warm-blooded animal affected by bacterial infections, which method comprises administering to said warm-blooded animal an effective amount of a compound of claim 1.

3. A pharmaceutical composition comprising an effective amount of a compound of claim 1 together with a pharmaceutically acceptable carrier.

4. A process for the preparation of bushrin which comprises cultivating Pseudomonas stutzeri (ATCC1607) or a mutant thereof under aerobic conditions, in a sterile liquid medium containing assimilable sources of carbon, nitrogen and inorganic anion and cation salts, until substantial confluence is obtained, extracting and isolating antibiotic bushrin from the said bacteria.

5. As claimed in claim 4 where the antibiotic bushrin is extracted in an organic solvent from the said bacterial growth medium.

6. As claimed in claim 5 where the organic solvent is ethyl acetate.

7. As claimed in claim 3 wherein said effective amount of said antimicrobial compound bushrin is from 0.01 to 5% by weight.
